(19) United States
(12) Patent Application Publication
(10) Pub. No.: US 2008/0065042 A1
(43) Pub. Date: Mar. 13, 2008

(54) ADJUSTABLE FIT PULL-ON GARMENT

(75) Inventors: Leigh E. Wood, Woodbury, MN (US); Rick M. Maveus, Wayzata, MN (US); Johann F. Petersen, Grevenbroich (DE)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/531,123

(22) Filed: Sep. 12, 2006

(51) Int. Cl. A61F 13/15

(52) U.S. Cl. 604/385.201; 604/392; 604/385.3; 604/385.11

(57) ABSTRACT

There is provided a disposable garment including a chassis having front and back waist sections. Two sets of side sections connect the front and back waist sections divided into a front side section and a back side section and define a continuous waist of the disposable absorbent garment. The leg openings are defined by the lower portion of the back side sections and the central crotch region of the chassis. The side sections form expandable waist and leg openings. A fastening element located a side section forms a refastenable closure when engaged with the chassis and with the back side sections creating the fitted waist and leg openings. The front side section is generally of a smaller width dimension so that it can be easily folded into the side of the diaper and does not form a part of either the fitted leg or waist openings.

### TECHNICAL FIELD

The present invention relates to a disposable absorbent garments suitable for use in various forms such as a disposable diaper, a disposable absorbent garment for incontinent patient, a disposable diaper cover and disposable training pants.

### BACKGROUND OF THE INVENTION

Disposable absorbent garments such as infant diapers or training pants, disposable infant swim pants, adult incontinence products, and other such products are well known in the art. The typical disposable absorbent garment is formed as a composite structure including an absorbent assembly disposed between a liquid permeable bodyside liner and a liquid impermeable outer cover. These components can be combined with other materials and features such as elastic materials and containment structures to form a product that is specifically suited to its intended purposes.

One type of disposable absorbent garment is the open style infant diaper, which is typically a flat, open-sided garment that is fitted about a child with the child laying down on top of the back portion of the garment. The front portion is folded onto the child and the back portion of the garment is typically releasably connected to the front portion of the garment by a closure mechanism or fastener on the back portion. The closure is engaged by pressure, which requires generally that the fastener by pressed into the front portion of the diaper pressing against the baby's tummy. Diapers are thus typically meant for use by children dependent upon a parent for assistance in putting on the absorbent garment. Such open style diapers are described in U.S. Pat. No. 3,860,003. One disadvantage of the open style diaper is that it can be difficult to put on infants who have learned to walk as they often do not like to be laid down for a diaper change. There is a conflict situation that arises causing difficulty for the caregiver who is changing the diaper and making it uncomfortable for the child. A further result is that the diaper is often not correctly fitted due to the diaper not being applied in the correct orientation and/or the fasteners being misplaced or misaligned on the diaper.

Pull-on style diapers, sometimes called pants style diapers, are also well known and several different designs are described in the patent art and are commercially available. For example, disposable training pants having front and rear portions fixedly attached together with side pane sections are known from WO-A-95129657 and WO-96131178, U.S. Pat. No. 4,205,679, U.S. Pat. No. 4,610,681, U.S. Pat. No. 4,938,753, U.S. Pat. No. 4,940,464, U.S. Pat. No. 5,171,239, and U.S. Pat. No. 5,843,068. These designs provide adjustability of the waistband size to a range of wearers due to various elastic arrangements of the side sections. This is the most common type of commercial execution and are put on and removed in much the same manner as elastic briefs where the waist regions are formed with substantially continuous waistbands. Such pull-on style of diapers are capable of being pulled up or down over the hips of the wearer and are most often used for specific applications, such as to assist in toilet training children, as the child can participate in the changing process. These training pants are easier, at least in part, to be correctly fitted compared to a diaper when being applied in that they are automatically in the correct orientation on the wearer when applied (the front back and sides are all aligned and in the correct locations). However, these types of training pant diaper have not always been able to achieve a close conforming fit to the wearer while still being able to expand enough to be pulled up and down over the hips of the wearer. For example, if training pants fit the waist of a wearer too loosely the pants can undesirably result in leaks, whereas if they are too tight they can tear or be difficult for the adult and particularly the child to put on. Such pull-on diapers also do not provide consumers with the ability to adjust the waist or leg fit to the wearer as in conventional open style diapers. Additionally such pull-on style diapers can be difficult to remove without pulling the diaper down, which can be messy, because the sides are sometimes difficult to tear open. One additional problem associated with such pull-on diapers is that there is no convenient way for them to be held in a rolled or folded configuration for disposal when soiled.

The lack of adjustability of pull-on style diapers can cause the diaper to fit too tightly when the child's waist is expanded, such as just after eating or if the wrong size is purchased. Many care givers prefer to have the ability to adjust the fit of the diaper and the tension exerted against the waist of the infant provided by open style diapers. Conventional pull-on diapers do not provide care givers this desired adjustability of the waist or leg fit. More specifically an aspect of the leg fit adjustability problem is that it can be difficult to get the feet and legs of the infant into the fixed diameter leg openings. This difficulty is acute when attempting to put such a conventional pull-on diaper on an infant without removing their shoes. With respect to waist adjustability the issue is mostly the waist tension in use. To overcome this lack of waist adjustability U.S. Pat. No. 5,531,732 teaches the use of a mechanical fastening tab permanently sealed to a fibrous web in the side section. This mechanical fastening tab can be used to adjust the waist fit of the pull-on diaper, however this does not address the issue of lack of adjustability of the leg openings.

Moreover, the removal of soiled pull-on pant type diapers has not always been completely satisfactory as they often do not provide adequate room for convenient removal and can be cumbersome to dissemble. To overcome this some commercially available pull-on diapers are provided with a frangible seam in the side section. In such products these seams can be opened to facilitate removal of the soiled diaper. Although this type of frangible seam makes it easier to remove a soiled diaper it is necessary that the force to open the seam be high enough to prevent the seam from being inadvertently torn when fitting the diaper onto an infant as the process of pulling the pull-on diaper over the hips of the infant puts significant stress on the side sections. An additional problem with such pull-on products is that they do not offer the care giver a mechanism for holding a soiled diaper in a folded or rolled configuration for disposal. To overcome this problem some such pull-on diapers are provided with separate elements designs specifically to hold the soiled diaper in a configuration for disposal. An example of a pull-on diaper with a disposal element is taught in U.S. Pat. No. 5,531,732.

Another type of pull-on diaper is designed with a refastenable attachment of the front and back portions of the side sections. This facilitates removal and can be used to allow the diaper to be opened to check for soiling and then reused if the diaper is not soiled. However it is difficult to reclose the refastenable attachment due to the need to properly align the refastenable parts of the side sections. Examples of such diapers are described in U.S. Pat. No. 6,849,067 and U.S. Pat. Appl. Nos. 2002/0173767 and 2004/016207.

To overcome the problems of these pull-on diapers the art describes various designs of pull-on diapers which are manufactured and provided in a pre-closed state, i.e. a prefastened state, ready to be pulled on for wearing, yet which comprises side fastenings which can be opened and reclosed.

Prefastened and refastenable disposable absorbent garments can provide advantages over either conventional open style or pull-on products. Open style products are generally flat and provided in an unfastened configuration, but include fasteners to secure the product about the wearer. Pull-on products, in contrast, have closed sides so that the product has a unitary waist opening and two leg openings. Prefastened and refastenable products can be applied and/or removed either like an open style diaper or like a pull-on diaper. For example, there may be times when it would be useful to apply the product like a pull-on diaper. In other instances, it might be more convenient to apply the product like an open style diaper, such as when there is a desire not to remove the child's shoes, but in this case the diaper is unfastened and cannot easily be refastened to reform the pull-on form. However because it is difficult to know when a particular mode of applying the garment will be needed, it is beneficial to have a garment that is adaptable to being used either as an open style diaper or as a pull-on product. This is preferable to keeping both types of garments available. A product that can be applied like either an open style diaper or a pull-on style diaper permits the interior of the product to be easily checked without having to pull the product downward. Such preclosed and refastenable diapers are described in U.S. Pat. No. 6,840,928, and U.S. Pat. Appl. Nos. 2002/0183712, 2002/0173765, 2002/0165518, and 2002/0165517.

Although such prefastened and refastenable products overcome some of the problems associated with open style and pull-on style diapers they do not solve the problem of the difficulty of using the diaper as a pull-on type diaper for putting the diaper on an infant without laying the infant down while overcoming the difficulty of getting the feet and legs into the leg openings, nor does it solve the difficulty that can be associated in pulling the diaper up over the hips of the infant.

Thus there remains a need for a disposable absorbent garment that can be put on like either open style diapers or pull-on style diapers and wherein when the garment is used as a pull-on style product the difficulties associated with getting the feet and legs into the leg openings is reduced, and wherein the fit about the waist of the wearer is readily adjustable. Additionally there remains a need for such a disposable absorbent garment wherein when the product is used as a pull-on product the difficulties associated with opening the product to check for soiling and for removing the garment in a convenient manner are minimized. Additionally there remains a need for such a disposable absorbent garment that is conveniently configured for disposal after use.

### SUMMARY OF THE INVENTION

The invention is directed at a disposable absorbent garment including a chassis having front waist section with an opposed back waist section and a crotch region disposed between and interconnecting the waist sections. The front and back waist sections are each configured to be positioned against the front and back, respectively, of a wearer of the absorbent article. Two sets of side sections extend between and connect the front and back waist sections. Each set of side sections is divided into a front side section and a back side section. The back side sections and the front and back waist sections of the chassis together define a continuous waist of the disposable absorbent garment. The leg openings are defined by the lower portion of the back side sections and the central crotch region of the chassis. The front side sections of the disposable absorbent garment are attached to the lateral edge of the front waist section of the chassis and a corresponding back side section. The back side sections are attached to the lateral edge of the back waist section of the chassis and the corresponding front side section, the front and back side sections are joined together to form expandable waist and leg openings. A fastening element located on, or attached to, the back side section is adapted to form a refastenable closure when engaged with the central region of the front waist section of the chassis creating contracted waist and leg openings. The back side sections are extensible, and generally elastic, to yield a snug fit of the garment about the waist and legs of the wearer. The front side section is adapted to be folded into the side of the diaper, and does fold into the side of the diaper, and does not form a part of either the leg or waist opening when the diaper is fitted to the wearer.

The front side sections are in one embodiment provided such that when the diaper is used by laying the child down after they step into the garment the back side sections can be extended to provide an adjustable fit while the applicator is holding the front section in place against the child or wearer.

The front side sections are in another embodiment provided such that the front side sections are narrower that the back side section at least adjacent the chassis. This raises the bottom edge of the expanded leg opening in the front side section, which increases the expanded leg opening at the front of the garment making the garment significantly easier to step into. In particular a narrow front side section raises the bottom edge of the front side section above the bottom edge of the back side section adjacent the chassis. This raises the top edge of the expanded leg opening in the front side section which more easily accommodates the front of a wearers foot, reducing the possibility that the front of the foot gets caught on the bottom edge of the front side section and reducing the need to get a child wearer to point their toes as they are putting on the garment.

Generally the expanded waist and leg openings of the disposable garment of the invention make it easier for a child to step into the garment than conventional pull-on diapers and the disposable garment of the invention can be fitted while standing or as in a conventional open style with the child then laid on their back. This allows for participation by the child and the full range of adjustability of a conventional open style diaper.

### BRIEF DESCRIPTION OF THE DRAWINGS

FIG. 1 is a perspective view of a garment of the invention with front side sections and back side sections in an expanded state.

FIG. 2 is a top view of the embodiment of FIG. 1.

FIG. 3 is a top view of the embodiment of FIG. 1 in a closed state.

FIG. 4a is a top view of a second embodiment of the invention in an expanded state.

FIG. 4b is a schematic top view of the FIG. 4a embodiment in a folded state prior to use.

FIG. 5a is a top view of another embodiment of the invention in an expanded state.

FIG. 5b is a schematic top view of the FIG. 5a embodiment in a folded state prior to use.

FIG. 5c is a schematic top view of the FIG. 5a type embodiment in another folded state prior to use.

FIG. 6 is a perspective view of another garment of the invention with front side sections and back side sections in an expanded state.

FIG. 7 is a cutaway perspective view of a garment of the invention as shown in FIG. 1 with front side sections and back side sections in an expanded state.

### DETAILED DESCRIPTION OF THE INVENTION

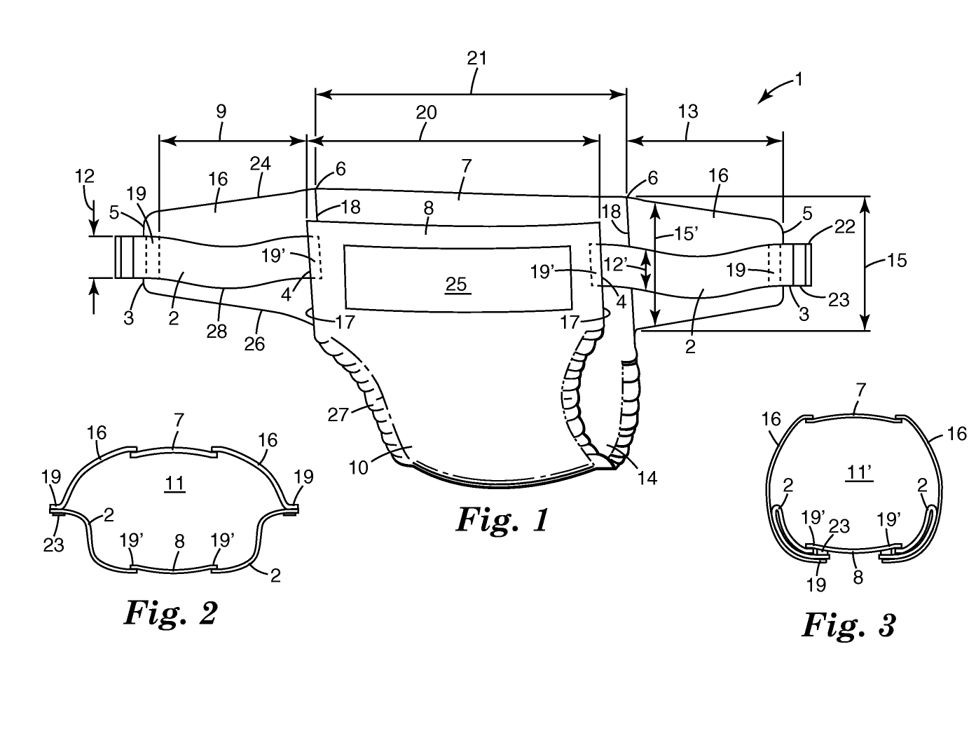

In a embodiment of the invention as shown in FIG. 1 fasteners 23 are attached to the back side sections 16 by use of a fastener tab 22 that enables the caregiver who is putting the garment on an infant to grasp the tab to conveniently pull a back side section 16 forward while positioning the fasteners 23 onto the central landing area 25 of the front waist section 8 of the chassis 10. The fastener tab 22 enables the fasteners 23 to extend outwardly from the continuous waist opening 11 when the waist opening is in the expanded configuration. When the fasteners 23 are releasably attached to the central landing area 25 of the front waist section 8 of the chassis 10 the fastener tab 22 becomes part of the fitted waist circumference opening 11′.

The circumference of the contracted or fitted waist opening 11′ is created by the combined circumferential dimensions of the front and back waist sections 8 and 7 of the chassis and the portions of the back side sections 16 that do not overlap with the front waist section 8. The circumference of the expanded waist opening 11 is created by the combined circumferential dimensions of the front and back waist sections 8 and 7 of the chassis and the front and back side sections 2 and 16. It is preferred that the ratio (the “fit ratio”) of this expanded waist opening circumference or open waist circumference (termed OWC as defined below) to an average fitted waist circumference (termed ARWC as defined below) be greater than about 1.25 or preferably 1.4 up to about 2. The larger fit ratios, of this expanded waist opening circumference (1.4 or higher) to the fitted or contracted waist circumference 11′ increases the ease of putting the garment on without catching the feet of the wearer. Higher fit ratios are made possible by using front side sections that are larger (greater in length but not necessarily wider, which they are preferably not) than the back side sections.

The back side sections 16 are formed such that they can create a fitted or contracted waist opening 11′ as well as fitted or contracted leg openings. This is generally done by having the top edge 24 of the back side section substantially aligned with the top edges of the front and/or back waist sections. This could be, for example, from 0 to 20 mm of the top edges of the front and/or back waist sections (7 and 8). The bottom edge 26 of the back side section 16 can be contoured so as to form the shape of the fitted or contracted leg opening to provide a contracted leg opening in conjunction with the central crotch region of the chassis. The central crotch region 14 is typically elasticized along the edge 27 to form an elasticized leg opening.

The front side section 2 can be of any suitable shape however as it does not form a part of the fitted waist or leg openings. The front side section 2 generally has a height 12′ that is narrower than at least the widest section of the back side section or narrower than the back side section at a given point spaced from where attached to the front and back waist sections (7 and 8). This provides a ratio of the front side section 2 height (termed FSSH as defined below) to the back side section 16 height (termed BSSH as defined below) of less than 0.7 and preferably less than 0.6. This saves on cost while also allowing the front side section 2 to be more easily folded into the side of the diaper when the back side section of the diaper is fitted to the wearer. A narrower front side section 2 also increases the expanded leg opening at the front of the garment making the garment easier to step into. A narrow front side section 2 will generally raise the bottom edge 28 of the front side section 2 above the bottom edge 26 of the back side section 16 adjacent the chassis or back waist section 8. This raises the top edge (bottom edge 28 of the front side section 2) of the expanded leg opening in the front side section 2 which more easily accommodates a wearers foot, reducing the possibility that the front of the foot gets caught on the bottom edge 28 of the front side section 2.

A section of the bottom edge 28 of front side section 2 adjacent the chassis, or front waist section 8, is preferably above a section of bottom edge 26 of the back side section 16 adjacent the chassis or back waist section 7. Another way to describe this is that when the garment is expanded and the front side section 2 is placed onto the back side section 16 as the diaper would be worn, in the area where they overlap, the back side section 16 bottom edge 26 extends beyond the front side section 2 bottom edge 28, at one or more sections. This allows a more secure fit to be created by the back side section 16 bottom edge 26 extending down to cover more of the buttocks and upper leg region of the wearer.

Another way to characterize the expanded leg opening is by looking at two partial perimeters, and the areas they circumscribe, of the expanded leg opening as shown in FIG. 7 and described in the example section below. The first partial perimeter 42 of the expanded leg opening is that encompassing the front side section (termed FPPA as defined below), with the second partial perimeter 43 encompassing the back side section (termed BPPA as defined below). Each partial perimeter is looked at using the fully extended the front and backside sections, as will be defined in the specific description of the drawings below. A perimeter area ratio (as defined herein) of the front side section partial perimeter area to the back side section partial perimeter area is at least 1.2 or 1.3 and in certain embodiments greater than 2 or even 3. This means there is significant more area for the front of the wearer's foot to get onto the garment than if the front and back side section partial perimeters 42 and 43 and their areas were the same.

The front side section 2 can be any suitable shape but a rectangular shape minimizes material usage while creating a larger expanded leg opening, or front partial perimeter area, for ease of stepping into by the wearer. The front side section 2 could be formed of any suitable material or laminate but is generally a soft flexible material and could suitably be a film or nonwoven web or laminates with nonwoven webs and/or films.

The front side section 2 in one embodiment is either longer than the back side section 16 or extensible, such as to allow the back side section 16 to expand when it is applied to the wearer. The front side section 2 is generally at least 10, 20 or even 50 percent longer (or expandable to this length) than the corresponding unextended back side section. The front side section 2 can be expandable by being formed in whole or in part with an expandable material or laminate such as an elastic web, a corrugated web, a micropleated web or the like. If expandable, the front side section 2 is generally expandable such that it can be longer that the corresponding unextended back side section 16 as described above. Preferable the front side section 2 is not entirely elastic if formed with an elastic material or web but has a discrete elastic segment of low modulus and high elongation, generally this elastic segment could be from 5 to 60 percent or 10 to 40 percent of the extent or length of the front side section 2. A frangible segment can also be provided on the front side section, which can be torn before placing the garment onto the wearer to use the garment as a true open style diaper, or to assist in removing the diaper.

Suitable materials for producing an elastic back side section 16 include, but are not limited to, stretch-bonded-laminate (SBL) materials, neck-bonded-laminate (NBL) materials, elastomeric films, elastomeric nonwovens, elastomeric foam materials, and/or the like. For example, suitable meltblown elastomeric fibrous webs are described in U.S. Pat. No. 4,663,220. Composite fabrics comprising at least one layer of a nonwoven material secured to a film or fibrous elastic layer are also desirable. Examples of suitable NBL materials are described in U.S. Pat. No. 5,226,992, issued Jul. 13, 1993 to Morman, the disclosure of which is incorporated by reference. The back side section is elastic at least in the direction of the waist circumference but can have elasticity in multiple directions.

The disposable absorbent garments of the invention are designed to come out of a package of the garments in a configuration wherein the fastening tab 22 or fastener 23 is in a storage position so that the caregiver can easily position the diaper with the waist in the fully expanded configuration to make it easy for an infant to step into the garment. Namely, the fastening tab 22 is not preattached to the central landing area 25. Once an infant's legs are placed through the expanded waist and leg openings the garment is pulled up into the final wearing position and the fastening elements or tabs 22 are used to tighten the fit of the garment about the waist and leg openings to provide a snug and comfortable contracted fit around the waist and upper legs of the infant. This can be done while the child/wearer is standing or they could be placed on their back and fitted as with an open style diaper. The garment is correctly positioned around the baby or wearer, as with a pull up type product (with the back, front and sides all correctly aligned), but both the waist and leg opening can be adjusted as needed, as with an open style diaper. The garment (e.g. a diaper) can be placed on the wearer while standing, or a child could be placed on their back and fitted. This is desirable as the garment is now correctly positioned on the baby as they have stepped into it as in a pull on diaper, and it can be rapidly closed around the baby to form a custom fit as with an open style diaper. An issue with open style diapers is always correctly positioning the baby on the diaper such that the diaper is centered on the baby and the back fasteners or fastening tabs are aligned with the fastening surface on the front waist section of the diaper so the back, front and sides are all aligned when the diaper is closed using the fasteners. This positioning function can be done with a baby's assistance with the invention garment rather than against their will.

In one embodiment of an article 1 of the invention, as shown in FIGS. 1-5c, the front side section 2 has a distal end 3, a proximal end 4, an unbonded length dimension 9 measured in the circumferential direction of the waist opening 11, and a height dimension 12 (although it can be trapezoidal in shape or other shapes). In this embodiment the back side section 16 has a distal end 5, a proximal end 6, a length dimension 13 measured in the circumferential direction of the waist opening 11, and a maximum height dimension 15, the back side section can be rectangular or trapezoidal or any other suitable shape. The proximal end 4 of the front side section 2 is bonded to the lateral edge 17 of the front waist section 8 of the chassis 10. The proximal end 6 of the back side section 16 is bonded to the lateral edge 18 of the back waist section 7 of the chassis 10. The distal end 3 of the front side section 2 is bonded to the distal end 5 of the back side section 16. These bonds preferably each include an overlap region 19, 19′ of the two materials forming the bond. For example, the proximal end 4 of the front side section 2 overlaps the lateral edge 17 of the front waist section 8 of the chassis 10 to form overlap region 19′ and the distal ends 3 and 5 of the front and back side sections 2 and 16 overlap at overlap region 19. The expanded waist opening 11 is determined by the length dimensions of the front and back side sections 2 and 16 and the front and back waist sections 8 and 7 of the chassis taking into account added together any overlap regions. In FIG. 1 the overlap region 19 of the front and back side sections are further provided with a fastener tab 22 which has an attached conventional fastener 23. In the alternative, as shown in FIG. 2, a fastener 23 could be directly applied at or adjacent the overlap region 19 on either the front side section (as shown) or an extending portion of the back side section (not shown). This overlap 19 of the front and back side sections could be eliminated if they were formed of a single material with a transition region between the two, where a fastener could be attached.

In the embodiments of FIGS. 1-5c the front side section 2 has a greater length, measured in the circumferential direction of the waist opening 11, than the back side section 16. This allows a person putting the garment on, e.g. a caregiver putting a diaper on an infant or an incontinent adult putting the garment on themselves, to hold a lateral edge 17 of the front waist section 8 of the chassis 10 (where the front side section attaches) in position against the body of the wearer with one hand while grasping the fastening tab 22, or fastener 23, and pulling the back side section 16 forward to place the fastener 23 in position on the central landing area 25 of the front waist section 8 of the chassis 10 to create the closure that holds the garment on the wearer. If the front side section 2 was inextensible and not longer than the back side section 16, the act of holding the lateral edge 17 of the front waist section 8 of the chassis 10 against the body of the wearer can cause the front side section 2 to prevent the back side section 16 from being elastically elongated in putting the garment on the wearer. The act of holding the lateral edge 17 of the front waist section 8 of the chassis 10 in place against the body of the wearer, when putting the garment on, is a natural motion of many people who use such garments to provide that the front waist section 8 is flat after fitting.

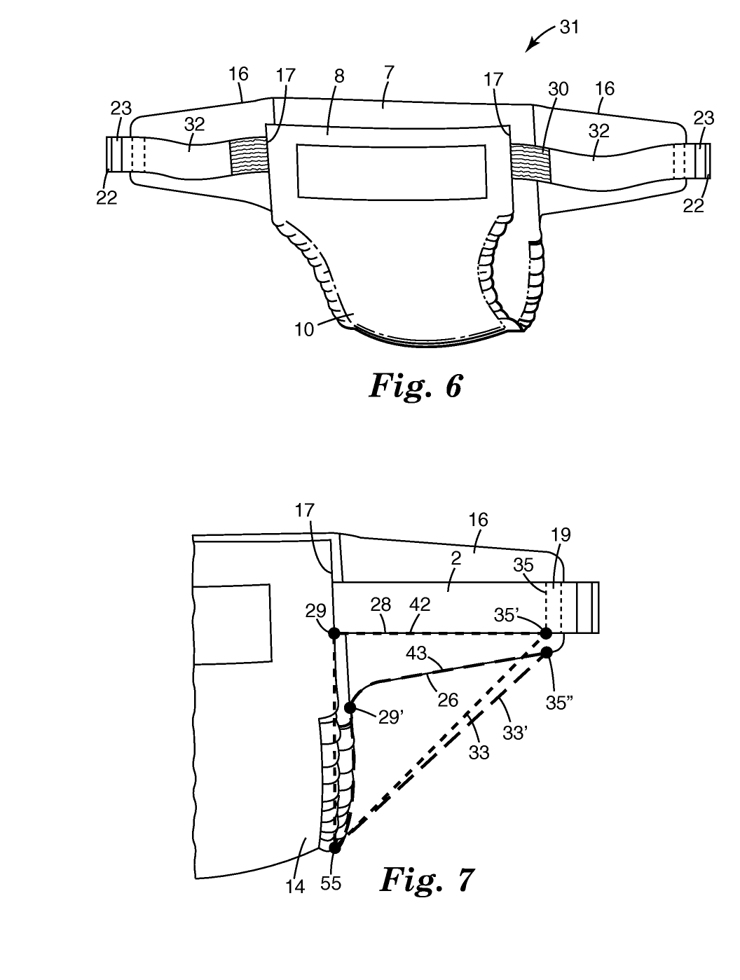

An additional embodiment, as shown in FIG. 6, comprises including a zone of elastic 30 in the front side section 32 to further limit the restriction to stretching of the back side section 16 by the front side section 32 when applying the garment 31. In such cases this elastic zone 30 of the front side section 32 can be located at any point between where the front side section 32 is bonded to the lateral edge 17 of the front waist section 8 of the chassis 10 and the front side section 32 is bonded to the back side section 16. The elastic zone 30 of the front side section 32 stretches elastically, in the circumferential direction of the waist opening, to further enable the back side section 16 to be elongated when applying the garment to the wearer.

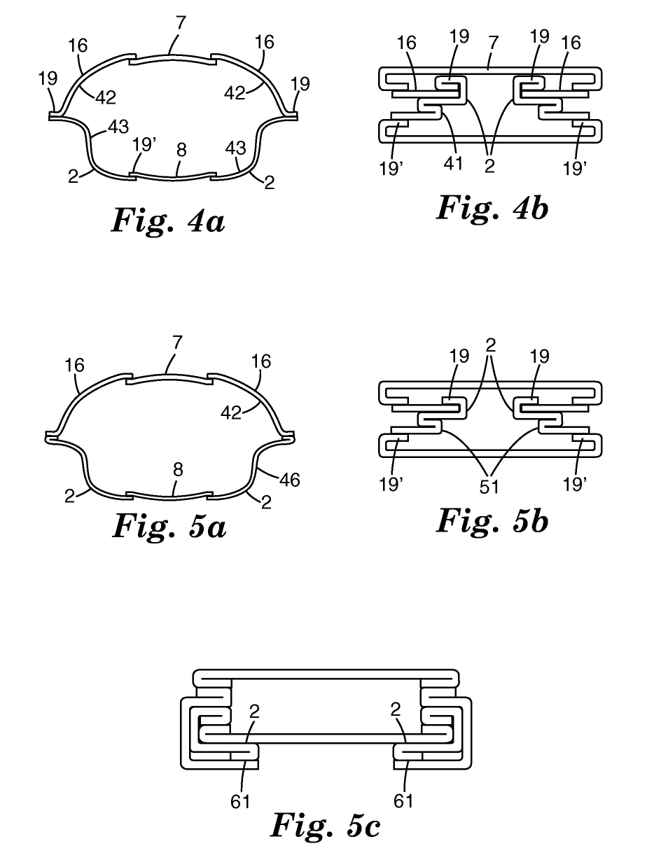

The garment can be folded prior to use as shown in FIGS. 4b, 5b and 5c. In FIGS. 4a and 4b the overlap region is as shown in FIGS. 1-3 where the inner faces 42 and 43 of the back side section 16 and the front side section 2 are bonded to form seamed overlap region 19. This creates seamed overlap region 19 that extends outward. This is desirable where the front and back side sections are supplied separately in line on a diaper line as they can easily be applied separately then they are laid upon each other and bonded prior to the chassis being folded into its final form. As the front side section 2 is longer than the back side section the front side section could be provided with one or more z folds 41 between the chassis overlap region 19′ and the side seam overlap region 19. In the embodiment of FIGS. 5a and 5b the front side section is likewise provided with one or more z folds 51 between the chassis overlap region 19′ and the side seam overlap region 19 where the front side section is folded onto an inner face of the chassis. In this embodiment however the side section overlap region 19 is formed by the outer face 46 of front side section 2 being bonded to the inner face 42 of the back side section 16 to form the side section overlap region 19. This creates a stronger bond as the bond is in shear mode if any stresses are encountered when the garment is applied to the wearer. FIG. 5c is the chassis of FIG. 5a type garment folded onto the outside face of the chassis provided with z folds 61 in the front side section 2. FIG. 5c is an alternative folding of the embodiment of FIG. 5a where the side seams are folded onto the outside of the diaper when packaged.

As opposed to traditional pull-on type diapers, wherein the elastic side sections must elastically stretch enough to allow the diaper to be readily pulled up over the hips and buttocks of the infant, as well as provide elasticity to comfortably accommodate movement of the infant and to maintain a snug fit of the diaper, in the present invention the back side sections do not need to be elastically elongated to pull the diaper up. The elastic back side sections only serve to comfortably accommodate movement of the infant and to maintain a snug fit of the diaper over a limited range of infant sizes and shapes. As such the degree of elastic stretch required is less and thus the amount of elastic material used in making the side sections of the garment of this invention can be significantly less than is used in making traditional pull-on diapers of a similar size.

The closure or fastening tabs 22 enable the person applying the garment to readily grasp the end of the tab while it is in a storage position (generally it could be folded over and temporarily attached to the front side section 2) and open it to a ready to use open position. In the storage configuration the fastener 23 faces the front side section 2 and may be removably engaged with the front side section 2. The fastener 23 can be any appropriate fastener material, including a hook material, a pressure sensitive adhesive, a cohesive closure material, or any other suitable fastener material. If the fastener is a hook material it is desired that the hooks lightly engage the front side section to hold the fastener tab in the storage configuration until the person applying the garment grasps the fastening tab and pulls it open, disengaging the hook form the front side section, into a ready to use configuration.

To facilitate grasping the fastener tab 22 it may be provided with a finger lift area. In one embodiment of the present invention the fastening tab is bonded to the distal end 5 of the back side section 16. This bonding can be achieved by any known method, including adhesive bonding, thermal bonding, sonic bonding, pressure bonding, or a combination of these methods. The fastener tab material can be any suitable material. Examples of suitable materials for the tab material include films, nonwovens, film-nonwoven laminates, paper, and similar materials. The fastener is bonded to the fastener tab material with any suitable bonding method, including adhesive bonding, thermal bonding, sonic bonding, pressure bonding, or a combination of these methods. For example when the fastener is a hook material adhesive bonding or a combination of adhesive bonding and sonic bonding or pressure bonding may be used to bond the hook material to the fastener tab material.

To further describe the invention an example of an embodiment of the invention as an infant diaper will be used to describe the garment but it is to be understood that with appropriate size adjustment the garment can be a disposable absorbent garment for incontinent adults, a outer cover for disposable absorbent garments that utilize disposable absorbent inserts, disposable swim ware for infants, and similar such disposable garments.

### EXAMPLES

As explained previously herein, in diapers of the present invention the three dimensional orientation of the leg opening is of importance; that is, the distances and relationships between various points of the front and back side sections that form part of the leg opening, and various points of the parts of the diaper chassis that form the remainder of the leg opening. Various measured parameters characterizing these relationships are presented in Table 1. With reference to these measurements, the following terminology is followed. The notation “open” means a configuration in which the diaper is held open or expanded, e.g. the position in which it would be placed upon the diaper wearer, or in which the diaper wearer would step into it. Such a position is represented in FIG. 2. The term “fitted” means a contracted, e.g. closed, configuration in which the diaper side sections have been placed upon and attached (e.g., by means of fastening tabs etc.) to the attachment area of the diaper backsheet; e.g., the configuration in which it would be worn by the diaper wearer. Such a configuration is represented in FIG. 3. The notation “vertical” means along the direction aligned with the torso of the diaper wearer. The notation “horizontal” means along a plane orthogonal to the vertical direction; that is, a plane parallel to the plane established by the waist of the diaper wearer. The term upper or top refers to that portion that would be uppermost along the vertical direction when the diaper is worn. The term lower or bottom refers to a position that would be lowermost when the diaper is worn.

One useful set of parameters are the partial perimeters. To measure the partial perimeter of the front side section of a given diaper, it was placed flat on a surface with the front side section 2 on top (in the configuration shown in the partial view of FIG. 7). The front side section 2 was positioned so it was flat and fully extended but not under tension. The lowermost point 55 of the leg opening at the crotch region 14 was allowed to reside at the position imparted by the structure of the diaper; that is, with no extension being placed upon it.

The front side section partial perimeter measurement originated at point 35′ at the lowermost point of bond area 35 at which the front and back side sections are joined (the bond or seam 35 typically occupies the proximal edge of the front side section/back side section overlap region 19 described previously). The distance was measured, along the lower edge of front side section 2, from point 35′ to point 29 at which front side section 2 joined the front waist section 8 of the diaper. The measurement continued from this point down to lowermost point 55 of the leg opening. The partial perimeter measurement was then completed by measuring theoretical straight line 33 (not corresponding to any physical edge or surface) from point 55 back to point 35′. The partial perimeter established by the front side section was thus that represented by (roughly triangular) dotted line 42 in FIG. 7. The area bounded by the front side section partial perimeter was calculated.

With regard to the partial perimeter of the back side section, the diaper was placed flat on a surface with the back side section 16 on top, with the back side section 16 flat and fully extended but not under tension. The lowermost point 55 of the leg opening at the crotch region 14 was allowed to reside at the position imparted by the structure of the diaper; that is, with no extension being placed upon it.

The back side section 16 partial perimeter measurement originated at point 35″, which is the point on the lower edge of the rear side section, directly vertically beneath seam 35. The distance was measured, along the lower edge of back side section 16, from point 35″ to point 29′ at which the back side section 16 joined the back chassis of the diaper. The measurement continued from this point down to lowermost point 55 of the leg opening. The partial perimeter measurement was then completed by theoretical straight line 33′ (not corresponding to any physical edge or surface) from point 55 back to point 35″. The partial perimeter established by the back side section 16 was thus that represented by (roughly triangular) dashed line 43 in FIG. 7. The area bounded by the back side section partial perimeter was calculated.

Measurements of the front and back side section partial perimeter areas are provided in Table 1. For each diaper, the ratio of Front Partial Perimeter Area (FPPA) to Back Partial Perimeter Area (BPPA) is also provided.

In order to more fully illustrate the invention, the front side section length 9 (FSSL) and back side section length 13 (BSSL) (as depicted in FIG. 1) are also included, as is the FSSL/BSSL ratio. In addition, the vertical front side section height 12 (FSSH) and back side section height 15′ (BSSH) are included, as is the FSSH/BSSH ratio. Vertical heights 12′ and 15′ were measured at points (on the front and back side section, respectively), that were 20 mm distal (that is, outwards along the side section) from the point at which the side section overlaps the chassis.

Another useful set of parameters is the waist circumference, in the opened and in the fitted position. One such measurement is the open waist circumference (OWC); that is, the length of the entire waist circumference of the diaper, as established by the chassis and the front and rear side sections, with the diaper in the open position (for example a position as represented in FIG. 2). This measurement was performed by placing the diaper flat, extending the front and rear side sections so that they were fully extended but not stretched, measuring the length of the waist along the top edge, and multiplying by two to obtain the circumference (In cases in which the front side section was longer than the rear, or vice versa, measuring the waist circumference in full extension necessitated folding the longer side section such that the fold occupied the most distal position from the diaper chassis. In this instance the length measured was to the distal fold, not to the joining seam between the two side sections). The tab and any portion of the front and/or back side section that was distal (outward) of the seam joining the front and rear side sections (hence did not contribute to the waist circumference) was not counted.

Another measurement was the minimum fitted waist circumference; that is, the length of the waist circumference with the diaper in the most tightly fitted condition (with the distal edges of the fastening tabs placed against each other on the front central landing area 25 of the diaper). This was obtained by placing the diaper flat with the fastening tabs placed as described above, extending the front and rear side sections so they were fully extended but not stretched, measuring the length of the waist along the top edge and multiplying by two. Again, no portion that did not contribute to the circumference was counted. In particular, in this configuration, portions of the front and/or rear side sections that were folded over the chassis, or over each other (such that they no longer contributed to the circumference) were not counted.

Another measurement was the maximum fitted waist circumference; that is, the length of the waist circumference with the diaper in the most loosely fitted condition (with the fastening tabs placed as far apart as possible, on opposing sides of the landing area 25), similar to the configuration shown in FIG. 3. With the diaper in this position, the measurement was obtained using the same procedure as for the minimum fitted waist circumference.

From these measurements were calculated the average fitted waist circumference (AFWC). Finally, the ratio of the open waist circumference to the average fitted waist circumference was calculated for each diaper. For each diaper, the open waist circumference, the average fitted waist circumference, and the OWC/AFWC ratio are present in Table 1.

Measurements were performed for the following:

D1—A diaper of the art was obtained (an open-style diaper available under the trademark PAMPERS CRUISERS (Size 5), from Proctor & Gamble, Cincinnati Ohio). This diaper was modified by connecting the as-manufactured front and rear side sections by joining them together at their proximal ends with a seam.

D2—A diaper of the art was obtained (an open style diaper available under the trademark PAMPERS CRUISERS (Size 5), from Proctor & Gamble, Cincinnati Ohio). This diaper was modified by removing the existing front side sections and replacing them with elastic front side sections with dimensions and properties as described in Table 1. The front and rear side sections were joined at their proximal ends with a seam, similar to that of D1.

D3—A diaper of the art was obtained (an open style diaper available under the trademark PAMPERS CRUISERS (Size 5), from Proctor & Gamble, Cincinnati Ohio). This diaper was modified by removing the existing front side sections and replacing them with inelastic front side sections with dimensions and properties as described in Table 1. The front and rear side sections were joined at their proximal ends with a seam, similar to that of D1.

D4—A diaper of the art was obtained (an open style diaper available under the trademark PAMPERS CRUISERS (Size 5), from Proctor & Gamble, Cincinnati Ohio). This diaper was modified by removing the existing front side sections and replacing them with front side sections with dimensions and properties as described in Table 1. The front side sections comprised an elastic section that was proximal to and attached via a seam to the diaper chassis, and comprised 26 mm of the total side section length, and an inelastic section that was distal to (and attached via a seam to) the elastic section, and which comprised 75 mm of the total side section length. The front and rear side sections were joined at their proximal ends with a seam, similar to that of D1.

TABLE 1
Diaper Measurements

Diaper

| | D1 | D2 | D3 | D4 |
|---|---|---|---|---|
| Front Side section | 40 | 67 | 99 | 101 |
| Length* | | | | |
| Back Side section | 60 | 55 | 58 | 59 |
| Length* | | | | |
| FSSL/BSSL*** | 0.67 | 1.21 | 1.70 | 1.71 |
| Front Side section | 63 | 45 | 40 | 40 |
| Height* | | | | |
| Back Side section | 105 | 105 | 105 | 105 |
| Height* | | | | |
| FSSH/BSSH*** | 0.60 | 0.43 | 0.38 | 0.38 |
| Front Partial | 1450 | 3470 | 4190 | 4690 |
| Perimeter Area** | | | | |
| Back Partial | 1210 | 1210 | 1210 | 1210 |
| Perimeter Area** | | | | |
| FPPA/BPPA*** | 1.19 | 2.87 | 3.46 | 3.88 |
| OWC* | 624 | 674 | 748 | 746 |
| AFWC* | 456 | 450 | 457 | 455 |
| OWC/AFWC*** | 1.4 | 1.5 | 1.6 | 1.6 |

*mm
**mm²
***dimensionless ratio

We claim:

1. A disposable absorbent garment including a chassis having a front waist section with an opposed back waist section and a crotch region disposed between and interconnecting the waist regions, two sets of side sections extending between and connecting the front and back waist sections, each set of side sections comprising an elastic back side section and a front side section, the elastic back side sections and the front and back waist sections of the chassis together define a contracted continuous waist of the disposable absorbent garment, leg openings of the disposable absorbent garment are defined by the lower portion of the elastic back side sections and the crotch region of the chassis, the front side sections are attached to a lateral edge of the front waist section of the chassis and joined with a corresponding back side section, the back side sections are attached to a lateral edge of the back waist section of the chassis and join with the corresponding front side section, the front and back side section when extended form expanded waist and leg openings, a fastening element associated with a terminal end of the back side section is adapted to form a refastenable closure when engaged with a central region of the front waist section of the chassis creating the contracted waist and leg openings, wherein the front side section allows the extension of the back side section and is adopted to be folded into the side on the diaper when the back side section is fitted to the wearer creating the contracted waist opening.

2. The disposable absorbent garment of claim 1 wherein the back side section and front side sections are separate materials joined at their terminal ends at an overlap region.

3. The disposable absorbent garment of claim 1 wherein a fastener tab is attached to a back side section.

4. The disposable absorbent garment of claim 1 wherein the ratio of the circumference of the OWC to the AFWC is greater than 1.25.

5. The disposable absorbent garment of claim 1 wherein the ratio of the circumference of the OWC to the AFWC is greater than 1.4.

6. The disposable absorbent garment of claim 1 wherein the ratio of the FSSH to the BSSH is less than 0.6.

7. The disposable absorbent garment of claim 1 wherein the bottom edge of the back side section is contoured.

8. The disposable absorbent garment of claim 1 wherein the ratio of the FSSH to the BSSH is less than 0.5.

9. The disposable absorbent garment of claim 1 wherein the ratio of the FPPA to the BPPA is greater than 1.2.

10. The disposable absorbent garment of claim 1 wherein the ratio of the FPPA to the BPPA is greater than 2.

11. The disposable absorbent garment of claim 1 wherein the ratio of the FPPA to the BPPA is greater than 3.

12. The disposable absorbent garment of claim 1 wherein the front side section length is at least 10 percent longer than the corresponding back side section length.

13. The disposable absorbent garment of claim 1 wherein the front side section length is at least 20 percent longer than the corresponding back side section length.

14. The disposable absorbent garment of claim 1 wherein the front side section length is at least 50 percent longer than the corresponding back side section length.

15. The disposable absorbent garment of claim 1 wherein the front side section is expandable such that it can be longer than the unextended corresponding back side section.

16. The disposable absorbent garment of claim 15 wherein the front side section is expandable by having an elastic segment over from 5 to 60 percent of the front side section length.

17. The disposable absorbent garment of claim 9 wherein the front side section is provided with a frangible segment.

18. The disposable absorbent garment of claim 8 wherein the garment is folded such that the front side section has one or more z folds between a chassis overlap region and a side section overlap region.

19. A disposable absorbent garment including a chassis having a front waist section with an opposed back waist section and a crotch region disposed between and interconnecting the waist regions, two sets of side sections extend between and connect the front and back waist sections, each set of side sections comprising an elastic back side section and a front side section each having a top edge and a bottom edge, the elastic back side sections and the front and back waist sections of the chassis together define a contracted continuous waist of the disposable absorbent garment, leg openings of the disposable absorbent garment are defined by the lower portion of the elastic back side sections and the crotch region of the chassis, the front side sections are attached to a lateral edge of the front waist section of the chassis and joined with a corresponding back side section, the back side sections are attached to a lateral edge of the back waist section of the chassis and join with the corresponding front side section, the front and back side section when extended form expanded waist and leg openings, a fastening element associated with a terminal end of the back side section is adapted to form a refastenable closure when engaged with a central region of the front waist section of the chassis creating the contracted waist and leg openings, wherein the ratio of the FSSH to the BSSH is less than 0.6.

20. The disposable absorbent garment of claim 19 wherein the back side section and front side sections are separate materials joined at their terminal ends at an overlap region.

21. The disposable absorbent garment of claim 19 wherein the ratio of the circumference of the OWC to the AFWC is greater than 1.25.

22. The disposable absorbent garment of claim 19 wherein the ratio of the circumference of the OWC to the AFWC is greater than 1.4.

23. The disposable absorbent garment of claim 19 wherein the ratio of the FSSH to the BSSH is less than 0.5.

25. The disposable absorbent garment of claim 19 wherein the ratio of the FPPA to the BPPA is greater than 1.2.

26. The disposable absorbent garment of claim 19 wherein the ratio of the FPPA to the BPPA is greater than 2.

27. The disposable absorbent garment of claim 19 wherein the front side section length is at least 10 percent longer than the corresponding back side section length.

28. A disposable absorbent garment including a chassis having a front waist section with an opposed back waist section and a crotch region disposed between and interconnecting the waist regions, two sets of side sections extend between and connect the front and back waist sections, each set of side sections comprising an elastic back side section and a front side section, the elastic back side sections and the front and back waist sections of the chassis together define a contracted continuous waist of the disposable absorbent garment, leg openings of the disposable absorbent garment are defined by the lower portion of the elastic back side sections and the crotch region of the chassis, the front side sections are attached to a lateral edge of the front waist section of the chassis and joined with a corresponding back side section, the back side sections are attached to a lateral edge of the back waist section of the chassis and join with the corresponding front side section, the front and back side section when extended form expanded waist and leg openings, a fastening element associated with a terminal end of the back side section is adapted to form a refastenable closure when engaged with a central region of the front waist section of the chassis creating the contracted waist and leg openings, wherein the ratio of the OWC to the AFWC is greater than 1.25.

29. The disposable absorbent garment of claim 27 wherein the ratio of the circumference of the OWC to the AFWC is greater than 1.4.

30. The disposable absorbent garment of claim 27 wherein the ratio of the FSSH to the BSSH is less than 0.6.

31. The disposable absorbent garment of claim 27 wherein the ratio of the FPPA to the BPPA is greater than 1.2.

32. The disposable absorbent garment of claim 27 wherein the ratio of the FPPA to the BPPA is greater than 2.

33. The disposable absorbent garment of claim 27 wherein the front side section length is at least 10 percent longer than the corresponding back side section length.

34. A disposable absorbent garment including a chassis having a front waist section with an opposed back waist section and a crotch region disposed between and interconnecting the waist regions, two sets of side sections extend between and connect the front and back waist sections, each set of side sections comprising an elastic back side section and a front side section, the elastic back side sections and the front and back waist sections of the chassis together define a contracted continuous waist of the disposable absorbent garment, leg openings of the disposable absorbent garment are defined by the lower portion of the elastic back side sections and the crotch region of the chassis, the front side sections are attached to a lateral edge of the front waist section of the chassis and joined with a corresponding back side section, the back side sections are attached to a lateral edge of the back waist section of the chassis and join with the corresponding front side section, the front and back side section when extended form expanded waist and leg openings, a fastening element associated with a terminal end of the back side section is adapted to form a refastenable closure when engaged with a central region of the front waist section of the chassis creating the contracted waist and leg openings, wherein the ratio of the FPPA to the BPPA is greater than 1.2

35. The disposable absorbent garment of claim 34 wherein the back side section and front side sections are separate materials joined at their terminal ends at an overlap region.

36. The disposable absorbent garment of claim 34 wherein the ratio of the circumference of the OWC to the AFWC is greater than 1.25.

37. The disposable absorbent garment of claim 34 wherein the ratio of the circumference of the OWC to the AFWC is greater than 1.4.

38. The disposable absorbent garment of claim 34 wherein the ratio of the FSSH to the BSSH is less than 0.5.

39. The disposable absorbent garment of claim 34 wherein the ratio of the FPPA to the BPPA is greater than 2.

40. The disposable absorbent garment of claim 34 the ratio of the FPPA to the BPPA is greater than 3.

41. The disposable absorbent garment of claim 34 wherein the front side section length is at least 10 percent longer than the corresponding back side section length.

42. A package of disposable absorbent garments the disposable absorbent garments including a chassis having a front waist section with an opposed back waist section and a crotch region disposed between and interconnecting the waist regions, two sets of side sections extend between and connect the front and back waist sections, each set of side sections comprising an elastic back side section and a front side section, the elastic back side sections and the front and back waist sections of the chassis together define a contracted continuous waist of the disposable absorbent garment, leg openings of the disposable absorbent garment are defined by the lower portion of the elastic back side sections and the crotch region of the chassis, the front side sections are attached to a lateral edge of the front waist section of the chassis and joined with a corresponding back side section, the back side sections are attached to a lateral edge of the back waist section of the chassis and join with the corresponding front side section, the front and back side section when extended form expanded waist and leg openings, a fastening element associated with a terminal end of the back side section is adapted to form a refastenable closure when engaged with a central region of the front waist section of the chassis creating the contracted waist and leg openings, wherein the front side section allows the extension of the back side section and is adopted to be fold into the side on the diaper when the back side section is fitted to the wearer creating the contracted waist opening wherein in the package the fastening element associated with a terminal end of the back side section is not engaged with a central region of the front waist section of the chassis.

43. The disposable absorbent garment of claim 42 wherein the front side section is folded into an inner face of the chassis.

44. The disposable absorbent garment of claim 42 wherein the front side section is folded onto an outer face of the chassis.

45. The disposable absorbent garment of claim 42 wherein the back side section and front side sections are separate materials joined at their terminal ends at an overlap region.

46. The disposable absorbent garment of claim 42 wherein the ratio of the circumference of the OWC to the AFWC is greater than 1.25.

47. The disposable absorbent garment of claim 42 wherein the ratio of the circumference of the OWC to the AFWC is greater than 1.4.

48. The disposable absorbent garment of claim 42 wherein the ratio of the FSSH to the BSSH is less than 0.5.

49. The disposable absorbent garment of claim 42 wherein the ratio of the FPPA to the BPPA is greater than 2.

50. The disposable absorbent garment of claim 42 the ratio of the FPPA to the BPPA is greater than 3.

51. The disposable absorbent garment of claim 42 wherein the front side section length is at least 10 percent longer than the corresponding back side section length.
